(19) United States
(12) Patent Application Publication
(10) Pub. No.: US 2024/0390174 A1
(43) Pub. Date: Nov. 28, 2024

(54) METHOD AND APPARATUS FOR SPINAL DISC DECOMPRESSION

(71) Applicant: VerteCore Technologies LLC, Crosby, MS (US)

(72) Inventors: Paul H. Leake, Crosby, MS (US); Paul R. Montalvo, Lewisville, TX (US)

(73) Assignee: VerteCore Technologies LLC, Crosby, MS (US)

(21) Appl. No.: 18/442,645

(22) Filed: Feb. 15, 2024

Related U.S. Application Data
(63) Continuation of application No. 15/092,545, filed on Apr. 6, 2016, now Pat. No. 11,931,283.
(60) Provisional application No. 62/144,958, filed on Apr. 9, 2015.

(51) Int. Cl. A61F 5/048; A61H 1/02

(52) U.S. Cl. CPC A61F 5/048; A61H 1/0218; A61H 1/0292; A61H 2201/1207; A61H 2201/1238; A61H 2201/1253; A61H 2201/1436; A61H 2201/1623; A61H 2201/1628; A61H 2201/1645; A61H 2201/165; A61H 2201/1664; A61H 2201/1697

(57) ABSTRACT

A portable spinal decompression device comprised of a top (thoracic) adjustable belt and bottom (lumbar) adjustable belt with a plurality of traction devices attached at set anchor points for applying traction forces to a body to facilitate spinal disc decompression and alleviate pain of users with spinal disorders. The portable device that fits various shapes and body sizes can be operated by a user without additional assistance and can be used while the individual is in numerous positions during his or her daily routine.

### CROSS-REFERENCES TO RELATED APPLICATIONS

This application claims the benefit of U.S. Provisional Application No. 62/144,958, filed Apr. 9, 2015, which is incorporated herein by reference in its entirety.

### FIELD OF THE INVENTION

The present invention is generally directed toward therapeutic decompressions. More specifically, it relates to an apparatus and method that promotes spinal disc decompression.

### BACKGROUND OF THE INVENTION

The basic principles of spinal traction have been offered by chiropractors, osteopaths, and other appropriately trained health professionals for many years. Both traction and decompression therapy are applied with the goal of relieving pain and promoting an optimal healing environment for bulging, degeneration or herniated discs. Pain relief is a result of creating a negative intradiscal pressure to promote retraction or repositioning of the herniated or bulging disc material and/or creating a lower pressure in the disc that will cause an influx of healing nutrients and other substances into the disc. Additionally, spinal decompression is used to relieve pressure on the spinal cord or one or more compressed nerve roots passing through the spinal column. Decompression of the spinal neural elements is a key component in treating spinal disorders such as radiculopathy, myelopathy and claudication.

Spinal decompression therapy involves stretching the spine, using traction or a motorized device, with the goal of relieving back pain. Devices that apply traction forces to the body to facilitate spinal disc decompression and provide relief to individuals in back pain are known in the art. However, the current decompression devices are bulky, difficult to operate, and require the user to stay in a single position in a single location, such as lying face down on a table. Also, the current devices are limited to a specific body type and thus, not available to individuals who may be suffering the most. Therefore, the need exists for a decompression device that is portable and allows the user to carry on normal daily activities while providing therapeutic relief to the user.

### SUMMARY OF THE INVENTION

The invention generally relates to a portable decompression device that provides traction and decompression therapy that relieves pain and promoting an optimal healing environment for bulging, degeneration or herniated discs. The device is comprised of a top (thoracic) adjustable belt and bottom (lumbar) adjustable belt with a plurality of traction devices attached at set anchor points for applying traction forces to a body to facilitate spinal disc decompression.

### BRIEF DESCRIPTION OF THE DRAWINGS

Further advantages of the invention will become apparent by reference to the detailed description of preferred embodiments when considered in conjunction with the drawings:

FIG. 1 is a front view of the device collapsed in neutral or start position.

FIG. 2 is a rear view of the device collapsed in neutral or start position.

FIG. 3 is a front view of the device extended for decompression with traction tensioner assembly handles in normal down position.

FIG. 4 is a rear view of the device extended for decompression.

FIG. 5 is a right side view of the device with the traction tensioner assembly as extended and with handle in downward neutral position.

FIG. 6 is a right side view of the device with traction tensioner assembly extended and handle in upright positive position.

FIG. 7 is a front view of the device in the process of decompressing by a user.

FIG. 8 is a side view of the device decompressed with traction tensioner assembly extended to provide decompression while the user is standing upright.

FIG. 9 is a rear view of the device with the user decompressed and resting on his or her side.

FIG. 10 is a side view of an embodiment of the device.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

This device solves the problems existing in the art by providing users a portable solution which they can operate while completing their normal daily activities. Unlike prior decompression devices which require users to remain in a single position without moving while receiving treatment from a second individual, this portable device can be operated and worn by users while driving, walking, working on a computer, sitting, cooking, etc. Furthermore, this invention provides pain relief to individuals with many different body shapes and sizes. For example, the device can be worn by children, athletes, and/or obese individuals who may be suffering the most from spinal disc pain.

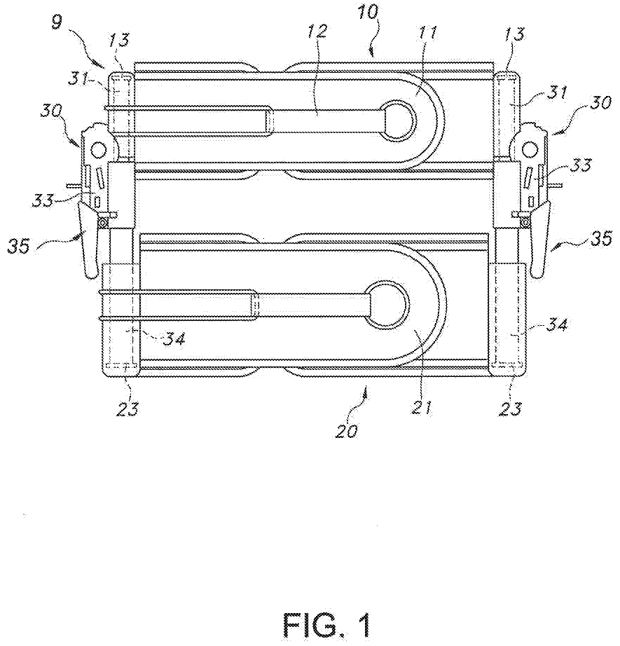

Referring to the drawings, as shown in FIG. 1, the device 9 consists of adjustable belts 10, 20 and a traction tensioner assembly 30 with a plurality of traction devices at set anchor points to allow for elongation and decompression of toothed rods 32 while on a user.

In the preferred embodiment of the device, the thoracic belt 10 is designed to encircle the torso and serves as the host for the top vertical anchor points 13 for extension or decompression. The lower lumbar belt 20 is designed to encircle the waist area and serves as the host for the lower vertical anchor points 23 for extension or decompression. Top vertical anchor points 13 and lower vertical anchor point 23 are attached or positioned into the top thoracic belt 10 and lower lumbar belt 20 respectively.

Both belts, the thoracic belt 10 and lumbar belt 20 are adjustable such that they can accommodate people of larger girths. In a preferred embodiment, the belts are adjoined by hook and loops which allow the position of the decompression rods 32 to be adjusted forward or back along the belts to accommodate a wide range of user sizes and body types and enhance the device's use as a treatment tool certain spinal disorders such as scoliosis.

The belts 10, 20 can be made of any material that has stretchable as well as fixed fibers which allow them to form, contour or wrap around the thoracic or chest region and waist regions. Preferably, the material is a breathable material such as nylon.

Also, the belts can be securely fastened and adjoined by mechanisms other than the hook and loop, such as clasps, buckles, snaps or any other type of adjoinment.

The belts may also include molded pieces, padding or inserts for greater comfort, tighter fits or more precise movement and adjustments. However, the belt design is not required for the present invention, and any other mechanisms capable of securing the traction tensioner assembly to the user are contemplated by this invention.

The decompression device operates by extending the distance between the two belts. Mechanisms for extending a rod 32 or exerting pressure between top and bottom anchor points 13, 23 such as hydraulic, pneumatic, electric or mechanical may be used in other embodiments of the present invention.

However, in the preferred embodiment, the traction tensioner assembly 30 serves as the connecting element between anchor points 13, 23 incorporated in the thoracic belt 10 and the lumbar belt 20 as well as the element to apply force utilized for extension or decompression. The traction tensioner assembly 30 allows the user to exert reasonable lift or force needed to decompress and then return to a neutral position by way of a user operated ratchet type actuator mechanism and incorporated release. When in use, the ratchet type actuator moves bi-directionally along the toothed rods 32 when lift or force is exerted upon the actuator handles 35 by the user.

To assemble the preferred embodiment of the device, the thoracic belt 10, containing at least two top anchor points 13 is inserted with the upper end 31 of two traction tensioner assemblies, one assembly for each anchor point inserted to form 90 degree angles. The lumbar belt 20, likewise containing at least two lower anchor points 23, is inserted with the lower portion of the same two traction tensioner assemblies 34, thus holding the two traction tensioner assemblies 30 on each side of the adjustable belts 10, 20 and parallel to each other.

The user first attaches the upper thoracic belt 10 around their chest, positioning close up in the axilla (armpit) area and adjusts said belt 10 for a tight fit using an elastic hook and loop closure of the thoracic inner wrap belt 11. The outer tension adjustment 12 can further be cinched to ensure a snug custom fit. The two top anchor points 13 attached to the belt on opposite halves are positioned snuggly within the recess of the axilla.

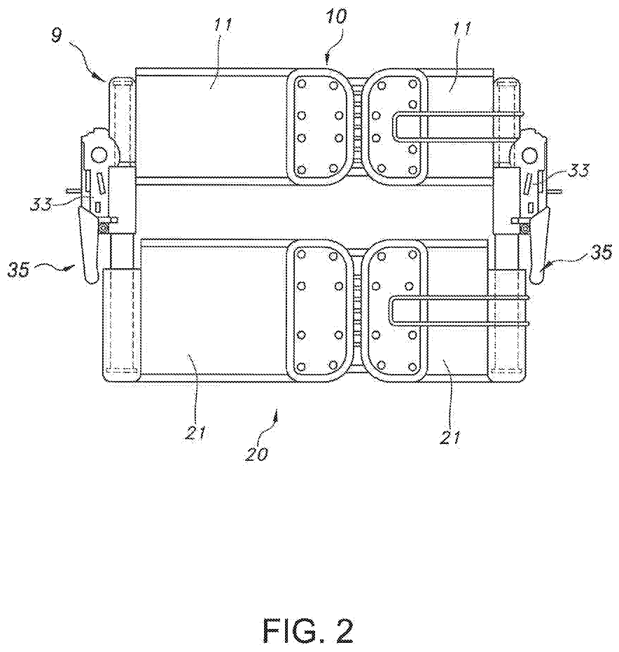

Next, the bottom lumbar belt 20 is placed around the waist and adjusted for a tight fit using the elastic hook and loop closure of the lumbar inner wrap belt 21. The user will note that the traction tensioner assembly 30 on each side runs from the axilla toward the hip and parallel to the body. As shown in FIGS. 1 and 2, once the user has finished attaching the device 9, it is compressed in neutral or start position.

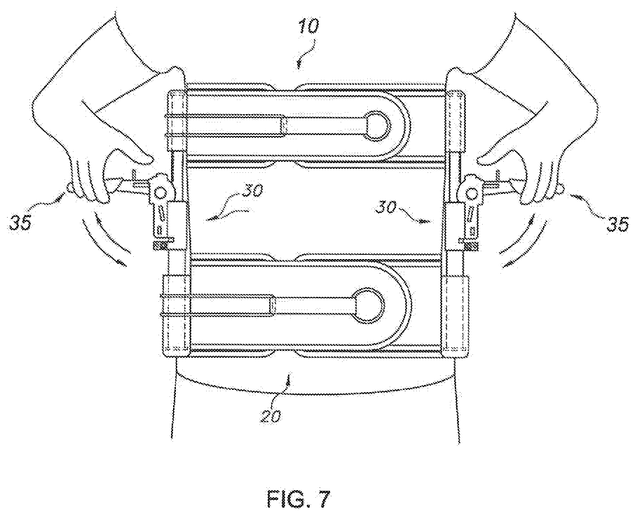

To extend the traction tensioner assembly 30 and begin decompression as shown in FIG. 7, the user simply grasps the incorporated actuator 33 with quick release handle 35 on the side of each traction tensioner assembly 30, raising and lowering the handles 35 in a back and forth ratchet action. An audible ratcheting sound confirms the extending action of the along the toothed rods 32 caused by stroking the handles 33.

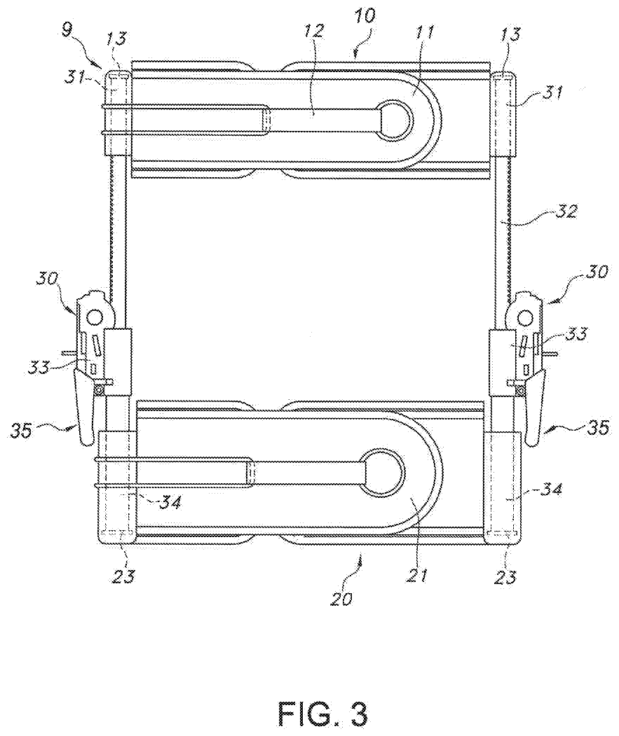

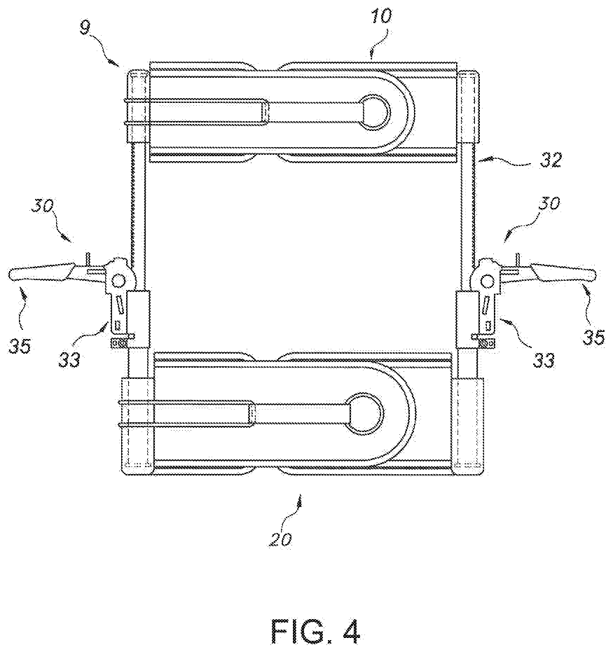

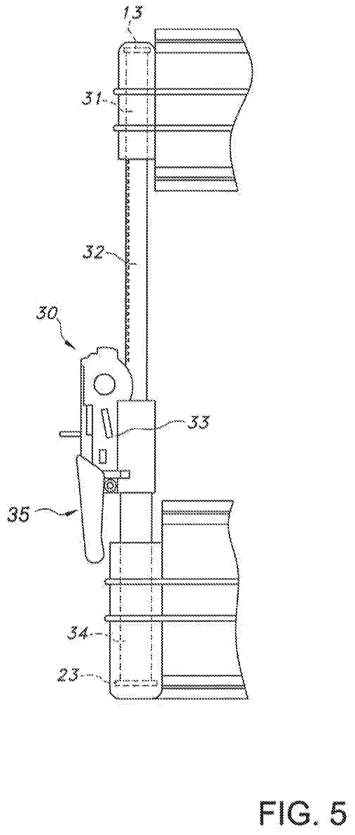

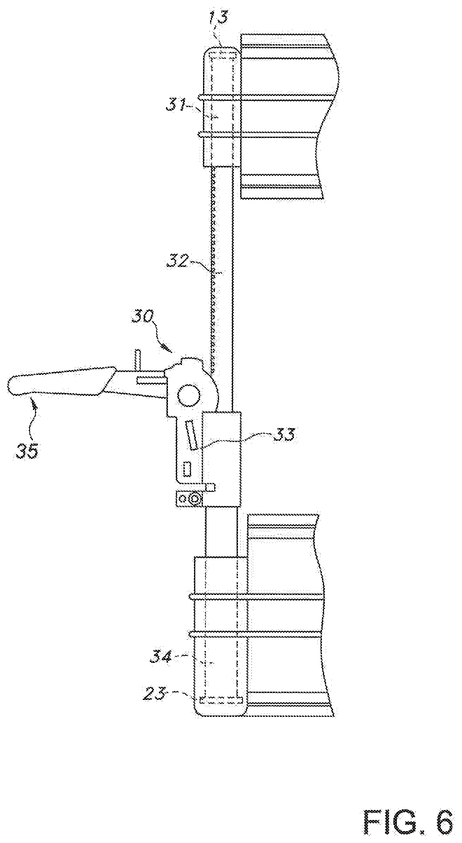

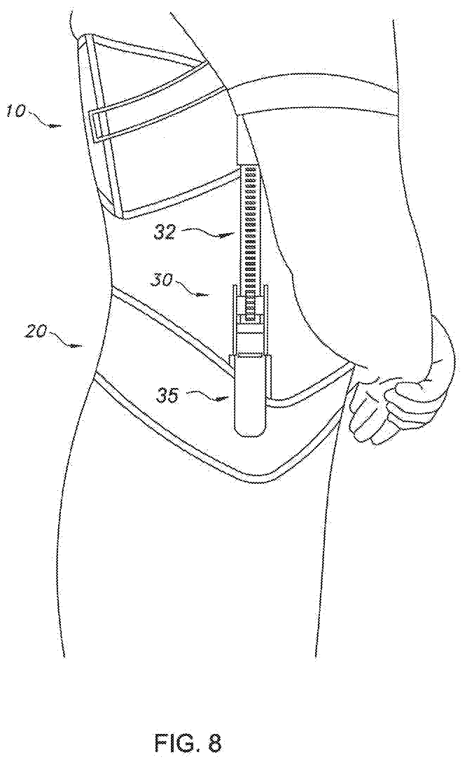

As the respective belts 10, 20 tighten against their anchor points 13, 23, decompression will begin as the spinal column is relieved of pressure. As shown in FIG. 4, when the handles are upright or in a positive position, the device is ready for the user to stroke the handles 35 down to apply more tension. Alternatively, as shown in FIGS. 3 and 10, the handles 35 are placed in a down position when the user feels they have reached a point of relief or decompression. After reaching this point of relief, as shown in FIG. 8, the users can continue their normal daily activities in in most any position such as standing, sitting, or laying.

Once the user feels relief or that they have sufficiently decompressed for one session, it is simply a matter of activating the quick release 35 on each traction tensioner assembly 30 actuator 33 to return the unit to a neutral position. The adjustable belts 10, 20 are removed in easy fashion, opposite from how they were put on and the entire unit is easily stored until the next use.

The anchor points 13, 23 which receive and transfer force for decompression can be fixed and utilized in various embodiments and of varying materials, including as illustrated in adjustable belts or in vests, single and dual point anchors which are transportable between utility style belts and various harnesses.

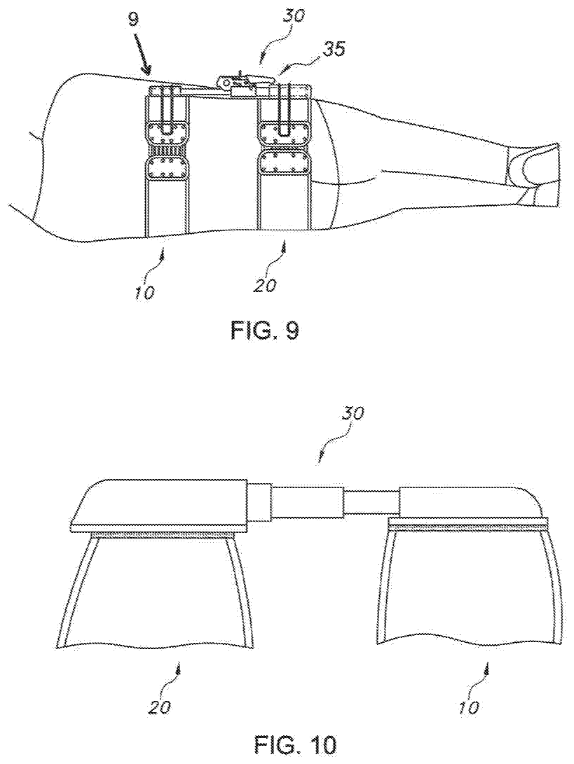

As shown in FIG. 9, a potential embodiment of the device 9 includes using rods 32 with telescoping stock to further reduce the height requirements of the rod 32 lengths when in a neutral starting position and further increase the range of individual users able to use the device 9.

A potential embodiment of the device provides an audible indicator when extension or decompression is being initiated, engaged or proceeding, with or without electronic readout for certain indicators such as pounds of force, time duration or length of extension.

The device can also be used to alleviate pain in different areas of the body. For instance, the device can be used to provide cervical traction to a user's neck.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

1-9. (canceled)

10. A portable spinal decompression device, comprising:
an inner thoracic belt positioned to completely encircle a chest of a user close to an axilla of the user, wherein the inner thoracic belt is formed of a stretchable material and adjoined by a first fastener mechanism that permits adjustment of the inner thoracic belt for a tight fit around the chest of the user;
a first outer tension adjustment cinched around the inner thoracic belt;
an inner lumbar belt positioned to completely encircle a waist of the user, wherein the inner lumbar belt is formed of a stretchable material and adjoined by a second fastener mechanism that permits adjustment of the inner lumbar belt for a tight fit around the waist of the user;
a second outer tension adjustment cinched around the inner lumbar belt;
at least one telescoping rod adjustable forward or back along the inner thoracic belt and the inner lumbar belt; and
a traction assembly configured to selectively extend the at least one rod for decompressing a spine of the user.

11. The device of claim 10, wherein the first fastener comprises hooks and loops for fastening the inner thoracic belt.

12. The device of claim 10, wherein the second fastener comprises hooks and loops for fastening the inner lumbar belt.

13. A method for use with a portable spinal decompression device, comprising:
completely encircling a chest of a user with an inner thoracic belt close to an axilla of the user, wherein the inner thoracic belt is formed of a stretchable material;
adjoining the inner thoracic belt via a first fastener mechanism, wherein the adjoining the inner thoracic belt comprises adjusting the inner thoracic belt for a tight fit around the chest of the user;
cinching a first outer tension adjustment around the inner thoracic belt subsequent to the adjoining the inner thoracic belt;
completely encircling a waist of the user with an inner lumbar belt, wherein the inner lumbar belt is formed of a stretchable material;
adjoining the inner lumbar belt via a second fastener mechanism, wherein the adjoining the inner lumbar belt comprises adjusting the inner lumbar belt for a tight fit around the waist of the user;
cinching a second outer tension adjustment around the inner lumbar belt subsequent to the adjoining the inner lumbar belt;
adjusting at least one telescoping rod forward or back along the inner thoracic belt and the inner lumbar belt; and
selectively extending the at least one rod for decompressing a spine of the user.

14. The method of claim 13, wherein the first fastener comprises hooks and loops for fastening the inner thoracic belt.

15. The method of claim 13, wherein the second fastener comprises hooks and loops for fastening the inner lumbar belt.
